(19) United States
(12) Patent Application Publication
(10) Pub. No.: US 2007/0071636 A1
(43) Pub. Date: Mar. 29, 2007

(54) Sterilization device

(76) Inventors: Jerald A. Bovino, Hollywood, FL (US); Janis Bovino, Bayside, NY (US)

Correspondence Address: EMCH, SCHAFFER, SCHAUB & PORCELLO CO, P O BOX 916, ONE SEAGATE SUITE 1980, TOLEDO, OH, 43697, US

(21) Appl. No.: 11/237,367

(22) Filed: Sep. 27, 2005

(51) Int. Cl. A61L 2/00

(52) U.S. Cl. 422/24; 250/461.1

(57) ABSTRACT

A device for sterilizing a surface having a light source positioned to direct a source of sterilizing light at said surface. The light directing sufficient sterilizing light at said surface to reduce the presence of infectious agents present on the surface to reduce the transmission of disease from contact with the surface.

### BACKGROUND OF THE INVENTION

The present invention relates to a device that can be utilized to sterilize a surface that is intended to be contacted by the public wherein contact transmission of infectious diseases can be significantly reduced or eliminated through the use of the sterilization device. More particularly, the invention utilizes ultra violet light or another source of pulsed light or radiation to sterilize a surface such as a control panel for an elevator that is designed and positioned to be in frequent contact with the general public.

Today we have a better appreciation of the spread of infectious diseases through contact transmission on surfaces that are generally utilized by the public. In almost all cities of the world there are surfaces that are constantly being contacted by the general public such as elevators control panels, door knobs, push plates on doors and similar structures that are designed to be used by the general public. Unfortunately, contact from the general public results in the contamination of such surfaces with infectious agents and these agents are capable of transmitting disease to other parties through contact with the surface. Recently there seems to be an increase in the incidences of transmission of such infectious diseases through contact transmission with such surfaces. With the advent of new infectious diseases, such as new strains of influenza, there is a definite need for an ability to reduce or eliminate the spread of infectious diseases through contact transmission. Accordingly, it is the object of the invention to provide a device that can be utilized to sterilize a surface that is generally used by the public to reduce or eliminate the spread of infectious disease by contact transmission whereby a non-infected person acquires the infectious disease from contact with the publicly available surface.

### SUMMARY OF THE INVENTION

The present invention is a device that utilizes a light source that is positioned to direct a source of sterilizing light at a surface. The light from the light source is efficient to sterilize the surface to reduce the presence of infectious agents on the surface to reduce the transmission of disease from contact with the surface

### BRIEF DESCRIPTION OF THE DRAWINGS

FIG. 1 is a front elevation view of the sterilization device of the present invention.

FIG. 2 is a side elevation view of the sterilization device shown in FIG. 1.

### DETAILED DESCRIPTION OF THE PREFERRED EMBODIMENT(S)

The invention is directed to a device which can be utilized to sterilize a surface that is frequently in contact with the general public. In particular, an ultra violet light or other form of pulsed light or radiation is directed at the surface to accomplish the sterilization. The device is particularly useful for applications such as elevator control panels or other surfaces that are frequently contacted by the general public during the utilization of the surface. The features of the invention will be more readily understood by referring to the attached drawings in connection with following detailed description.

Infectious diseases are spread by several methods. Epidemiologists understand that one of the most important forms is contact transmission for the spread of infectious diseases. In contact transmission it is necessary for the person to actually touch the infectious agents to spread the infection or disease. Contact transmission can be accomplished by direct contact which involves immediate contact between two people or indirect contact where an object that is contaminated by the touch of one person, spreads the infectious disease when another person touches the contaminated surface. Examples of infectious diseases that can be spread by contact transmission include the norovirus recently identified as causing cruise ship illnesses, various forms of the epidemic keratoconjunctivitis, many strains of influenza, herpes and other infectious diseases. Epidemics of infectious diseases can spread particularly quickly in high density or crowded places because the people who are infected transmit the infectious disease to various surfaces that they contact in public settings and these same surfaces are then contacted by people who are susceptible to contacting the infectious disease. One of the most effective means to reduce the incidence of contact transmission for infectious diseases is the frequently washing of an individuals hands to remove or sterilize the infectious diseases that are picked up through contact in public facilities. However, there are many frequently used public contact surfaces that are encountered on a daily basis such as door handles, push bars on doors, elevator buttons and similar surfaces that are intended to be utilized on a frequent basis by the general public. Such surfaces can be particularly effective in providing a source for contact transmission of infectious diseases.

One example of the type of public surface that is frequently involved in contact transmission of infectious diseases is the control panel or elevator buttons that are found in elevators. It is necessary to engage the buttons or contact surface in the elevator to reach the desired destination and the elevators are often used with a frequency that does not accommodate the frequent washing of the users hands or the elevators may be in a location that is not convenient to a facility that allows the user to wash their hands immediately after coming into contact with the elevator control panel. The details of the invention will be described with respect to an elevator control panel as this is a particularly significant publicly utilized surface that can be involved in the contact transmission of infectious diseases. The elevators are also frequently crowded with many people touching or otherwise placing infectious diseases on the control panel whereby the infectious diseases can be spread by contact transmission. Such elevator control panels or buttons are well recognized in the medical community as a very effective method for spreading infectious diseases caused by spores, fungi, molds, bacteria and viruses through contact transmission. However, it should be understood that the invention can be utilized with other surfaces to sterilize the surface to reduce the transmission of infectious diseases.

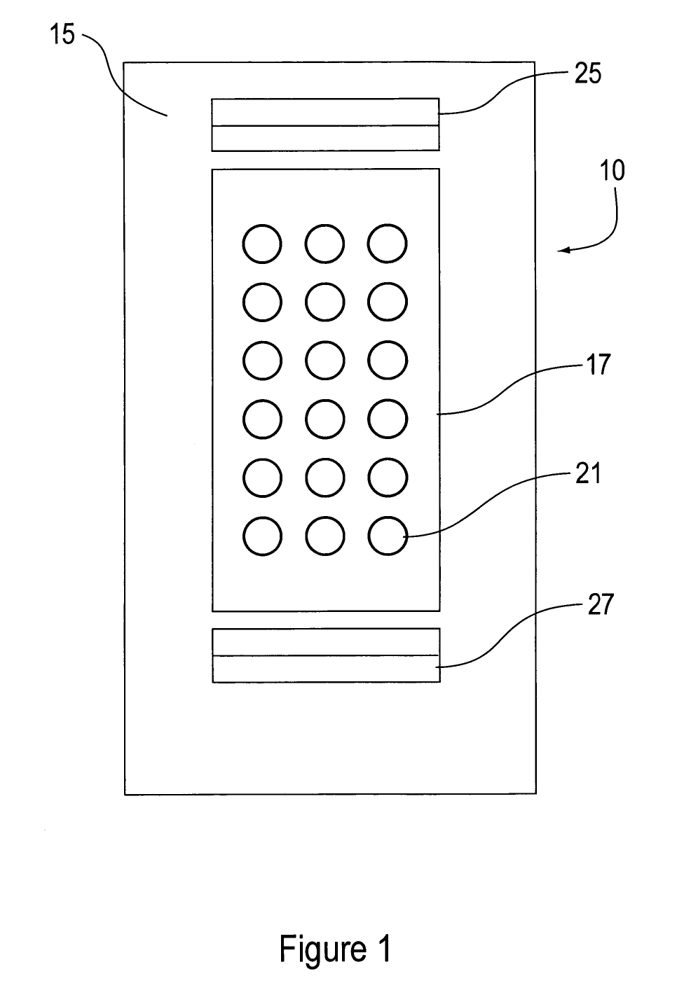

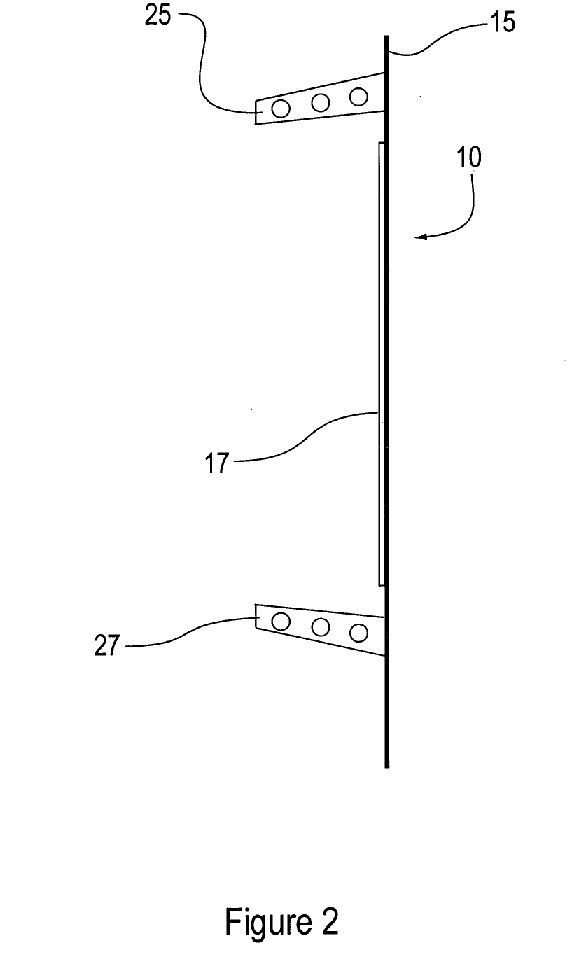

FIGS. 1 and 2 shows an elevator 10 having a wall 15 with a control panel 17 that contains a plurality of buttons 21 that are engaged to direct the elevator to the desired floor in a building. Positioned adjacent one end of the control panel 17 as a light source 25 that is used to direct ultra violet light or another form of pulsed light or radiation on to the control panel 17 and the elevator buttons 21. As shown in FIG. 2 the ultra violet light from the light source 25 is directed in a manner that it contacts the entire surface of the control panel 17. A second light source 27 can be positioned adjacent the opposite end of the control panel 17 whereby the second light source also directs ultra violet light onto the control panel 17. The second light source 27 can be utilized to ensure that the entire surface of the control panel 17 is contacted by the ultra violet light. The ultra violet light from the lights sources 25 and 27 should be directed continuously at the control panel 17 at least during the time period that the control panel is utilized to activate the elevator. If the elevator is taken out of service for some period of time during non peak usage periods the light sources 25 and 27 could be turned off or deenergized to reduce the cost of operating the light sources.

Ultra violet lights are used to sterilize work places and tools in biological laboratories and medical facilities. Low pressure mercury discharge lamps emit ultra violet light at a level that is very effective for acting as a germicidal agent. The ultra violet light can also act upon the thymine molecules on the DNA of an infectious disease to create defects in the DNA of the infectious disease. If the ultra violet light creates enough defects in the DNA of the organism, the organism's ability to replicate is inhibited and the effectiveness of the organism is significantly reduced. Although ultra violet light has been specifically mentioned as the sterilization medium it should be understood that other forms of intense pulsed light, ionizing or non-ionizing radiation can be used in the sterilization process and directed at the control panel 17 from the light source 25 and 27. The sterilization device of the present invention can be incorporated into a new facility or elevator as part of an infectious disease control program. Or the device of the present invention can be utilized as an after market device that is retrofitted to sterilize surfaces that are intended to be contact by the general public. The sterilization light that is continuously directed at the control panel will significantly reduce the presence of infectious agents and dramatically decrease the incidence of contact transmission of disease that takes place in public places.

The above detailed description of the present invention is given for explanatory purposes. Various modifications and substitutions, other than those cited, can be made without departing from the scope of the invention being defined solely by the appended claims.

We claim:

1. A device for sterilizing a surface comprising:
a light source positioned to direct a source of sterilizing light at said surface, said light directing sufficient sterilizing light at said surface to reduce the presence of infectious agents present on the surface to reduce the transmission of disease from contact with the surface.

2. The device of claim 1 wherein the light source directs ultra violet light at the surface.

3. The device of claim 1 wherein the light source directs ionizing or non-ionizing radiation at the surface.

4. The device of claim 1 wherein the light from the light source is pulsed intense light.

5. The device of claim 1 wherein the light source directs light at the surface during the time period that the surface is accessible by the public.

6. The device of claim 1 wherein the surface is a control panel for an elevator.

7. The device of claim 1 wherein the light is a low pressure mercury discharge lamp.
